(19) United States
(12) Patent Application Publication
(10) Pub. No.: US 2026/0182901 A1
(43) Pub. Date: Jul. 2, 2026

(54) METHOD AND SYSTEM FOR IDENTIFYING ABLATION LINES

(71) Applicant: UNIVERSITEIT GENT, Gent (BE)

(72) Inventors: Nele VANDERSICKEL, Gent (BE); Sander HENDRICKX, Gent (BE); Robin VAN DEN ABEELE, Gent (BE)

(73) Assignee: UNIVERSITEIT GENT, Gent (BE)

(21) Appl. No.: 19/130,659

(22) PCT Filed: Dec. 1, 2023
(86) PCT No.: PCT/EP2023/083936

§ 371 Date: May 16, 2025

(30) Foreign Application Priority Data
Dec. 2, 2022 (EP) 22211082.7
Feb. 23, 2023 (EP) 23158092.9
Sep. 18, 2023 (EP) 23197868.5

(51) Int. Cl. A61B 5/367; A61B 5/00

(52) U.S. Cl. CPC A61B 5/367; A61B 5/7264

(57) ABSTRACT

The present invention is in the field of cardiac arrhythmia. In particular, the present invention relates to systems and methods for identifying ablation lines, which can then be used to correct cardiac arrhythmia.

### TECHNICAL FIELD

The present invention is in the field of cardiac arrhythmia. In particular, the present invention relates to systems and methods for identifying ablation lines, which can then be used to correct cardiac arrhythmia.

### BACKGROUND

Cardiac arrhythmia is a condition in which the heartbeat is irregular and/or has an abnormal frequency, e.g., in which an abnormal resting heart rate can be observed. It is known in the art that such arrhythmias can be caused by an abnormal electrical conduction in the heart or near the heart, i.e., by abnormalities in the action potential impulse propagation system that drives the heart contraction in a synchronized manner. Atrial tachycardia (AT), in particular, is a regular arrhythmia which causes a fast heart rate.

In general, an AT can have two possible mechanisms: anatomical reentry or focal activity. To describe anatomical reentry, the anatomy of the left and the right atrium present us 3 natural openings or holes. For the left atrium, the three openings are the mitral valve (MV), the left pulmonary veins (LPV) and the right pulmonary veins (RPV). For the right atrium, the openings are the tricuspid valve (TV), the inferior vena cava (IVC) and the superior vena cava (SVC). Besides these natural obstacles, it is possible that the patient has additional non-conductive tissue in the form of scar tissue, which creates additional obstacles in the atria. Therefore, anatomical reentry can be divided in two types: (1) one can have macro-reentry around one of the 6 anatomical larger obstacles as described above namely the MV, LPV, RPV, TV, IVC, SVP, and (2) reentry can occur around scar tissue. When the scar tissue is small, this is often called micro-reentry. Focal AT is defined as atrial activation starting rhythmically at a small area (focus) from which it spreads out centrifugally and without endocardial activation over significant portions of the cycle length.

Currently, if medication does not solve the issue, a cardiac ablation may need to be performed. Cardiac ablation uses heat or cold energy to create tiny scars in the heart to block irregular electrical signals and restore a typical heartbeat. Cardiac ablation is most often done using catheters inserted through the veins or arteries; or may be performed during cardiac surgery. However, before starting the procedure, suitable ablation lines need to be identified.

In a typical procedure, an electrophysiologist (EP) will try to induce the arrhythmia so it can be mapped. The 3 main mapping systems are RHYTHMIA (Boston Scientific), CARTO (Biosense Webster), and Ensite (Abbott). During the mapping procedure, a large number of electrode recordings may be sequentially recorded, thereby providing a full map of the atrial tachycardia. As the atrial tachycardia is regular, one could use the same recording as if it had been taken at a single time. From each recording, an intra-cardiac signal may be recorded, while knowing the exact spatial location (x,y,z) of that recording. From the intra-cardiac signal, local activation times are extracted, which, in combination with the spatial location, give rise to a colour map. This colour map shows the wave propagation of the atrial tachycardia and forms the basis for the ablation strategy. In addition to this colour map, many algorithms have been added to augment the colour map, for an easier interpretation of the colour map, like visualizing the atrial activation wavefronts with velocity vectors. It is important to be able to analyse the mechanism of the AT to decide the optimal ablation line. In case of anatomical reentry, the EP will connect the obstacle around which the reentry occurs to another obstacle, so the reentry path is completely blocked. In case of a focal source, this source itself will be ablated.

Each mapping system may have added additional features to find the source of the arrhythmia more easily. However, the final diagnosis of the source of the arrhythmia still depends on the interpretation of the electrophysiologist. Moreover, giving the same map to different electrophysiologists may lead to different diagnosis of the atrial tachycardia, which is problematic. In addition, given a certain mechanism of the atrial tachycardia, different electrophysiologists may also propose a different set of ablation lines, depending on their personal preferences or on the school of origin. In addition, very often a patient needs to be remapped during a single procedure as ablation often converts an AT into a slower AT. Remapping is time intensive, and therefore increases the time the patient needs to be on the table under sedation, while the EP needs to spend more time in the cath lab.

Accordingly, there is a need for systems and methods that provide an improved indication to the electrophysiologist of where the ablation should occur. There is also a need for systems and methods that provide an objective indication to the electrophysiologist of where the ablation should occur. There is also a need for systems and methods that provide an indication to the electrophysiologist of where the ablation should occur while simultaneously minimising scar tissue.

### SUMMARY OF THE INVENTION

The present invention overcomes one or more of these issues. Preferred embodiments of the present invention overcome one or more of these issues, for example by:
providing an objective diagnosis that is independent of the electrophysiologist;
providing a minimal set of anatomical objects that need to be connected by ablation lines;
allowing to minimize the total area to be ablated;
allowing to reduce the number of maps which need to be recorded; and/or,
preventing additional ablation.

The systems and methods of the present invention provide a tool that aims to give a unique classification for every mapped atrial tachycardia. As this classification is unique, and may be fully performed automatically, systems and methods of the present invention are operator independent. The systems and methods of the present invention also propose an ablation strategy by identifying which parts of the heart need to be connected. However, in some case, there are multiple possibilities, and the electrophysiologist may still choose their preferred strategy. Mathematically, in case there are multiple possibilities, they are equivalent.

In a first aspect, the present invention relates to a method, preferably a computer-implemented method, for identifying possible ablation lines. The method preferably comprises the steps of:
a) receiving spatiotemporal electrophysiological data of a subject's heart;
b) converting the spatiotemporal electrophysiological data into converted data; preferably wherein the converted data comprises a directed graph;
c) identifying one or more reentry loops from the converted data; preferably wherein the converted data comprises a directed graph;
d) identifying one or more suppressed loops from the converted data;
preferably wherein the converted data comprises a directed graph; and,
e) identifying a set of one or more possible ablation lines that terminate the one or more reentry loops and the one or more suppressed loops.

Steps a) and b) may be considered pre-processing steps and may have been previously performed. Therefore, in the first aspect, the present invention also relates to a method, preferably a computer-implemented method, for identifying possible ablation lines, the method comprising the steps of:
ab) receiving converted spatiotemporal electrophysiological data of a subject's heart; preferably wherein the converted data comprises a directed graph;
c) identifying one or more reentry loops from the converted data; preferably wherein the converted data comprises a directed graph;
d) identifying one or more suppressed loops from the converted data; preferably wherein the converted data comprises a directed graph; and,
e) identifying a set of one or more possible ablation lines that terminate the one or more reentry loops and the one or more suppressed loops.

In some preferred embodiments, the converted data comprises a directed graph, preferably obtained using directed graph mapping or DGM.

In some preferred embodiments, in step d) a suppressed loop is defined as a partially complete loop with a threshold of completion, whereby the threshold is preferably at least 50%.

In some preferred embodiments, step d) comprises visual identification of the suppressed loops by a user. In some preferred embodiments, step d) comprises automatic identification of the suppressed loops by a computer.

In some preferred embodiments, step e) comprises classifying the identified loops prior to identification of the ablation lines.

In some preferred embodiments, step e) comprises the step of identifying an isthmus common to a reentry loop and a suppressed loop.

In some preferred embodiments, step e) comprises the step of identifying an ablation strategy comprising a minimal total ablation length. In some preferred embodiments, step e) comprises the step of identifying a minimal number of ablation lines that terminate all identified reentry loops and suppressed loops. In some preferred embodiments, step e) comprises the step of imposing constraints of areas that are not to be ablated.

In some preferred embodiments, the method comprises the step of consulting a proposed ablation strategy in a topological database.

In a second aspect, the present invention relates to a system or device for identifying ablation lines. The system or device preferably comprises:
an input unit configured for receiving converted spatiotemporal electrophysiological data of a subject's heart;
a topological feature analyser for determining features in the converted data;
   wherein the topological feature analyser is configured for identifying reentry loops as well as suppressed loops.

The system or device alternatively comprises:
an input unit configured for receiving spatiotemporal electrophysiological data of a subject's heart;
a mapping unit and a directed graph generator configured for converting the spatiotemporal electrophysiological data in a directed graph; and,
a topological feature analyser for determining features in the directed graph;
   wherein the topological feature analyser is configured for identifying reentry loops as well as suppressed loops.

In some preferred embodiments, the system or device according to the second aspect of the invention, and (preferred) embodiments thereof, is configured to perform the (computer-implemented) method according to the first aspect of the invention, and (preferred) embodiments thereof.

In a third aspect, the present invention relates to a computer program product directly loadable into the internal memory of a computer, or a computer program product stored on a computer readable medium, or a combination of such computer programs or computer program products, configured for performing a computer-implemented method according to the first aspect of the invention, and (preferred) embodiments thereof.

In a fourth aspect, the present invention relates to use of the computer-implemented method according to the first aspect of the invention, and (preferred) embodiments thereof, or of the system or device according to the second aspect of the invention, and (preferred) embodiments thereof, for a for a subject suffering from atrial tachycardia, ventricular tachycardia, or atrial fibrillation; preferably atrial tachycardia.

In a fifth aspect, the present invention relates to computer readable storage medium comprising a topological database, the topological database preferably comprising ablation strategies corresponding to identified reentry loops and suppressed loops, suitable for use in the computer-implemented method according to the first aspect of the invention, and (preferred) embodiments thereof.

(Preferred) embodiments of the first aspect of the invention are (preferred) embodiments of the second, third, fourth, and/or fifth aspect of the invention, and vice versa.

Particular and preferred aspects of the invention are set out in the accompanying independent and dependent claims. Features from the dependent claims may be combined with features of the independent claims and with features of other dependent claims as appropriate and not merely as explicitly set out in the claims. These and other aspects of the invention will be apparent from and elucidated with reference to the embodiment(s) described hereinafter.

### DESCRIPTION OF THE FIGURES

The following description of the figures of the invention is only given by way of example and is not intended to limit the present explanation, its application, or use. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

FIG. 1 illustrates the various steps in directed graph mapping (DGM): (A) identification of neighbours, (B) construction of a directed graph, (C) construction of a merged graph, (D) calculation of the DGM nodes, (F) calculation of the DGM wavefront, (E) calculation of the DGM loop-band.

FIG. 2 illustrates 9 different options with 2 loops consisting of at least 1 reentry loop. The reentry loops are indicated by a full oval around a hole, the suppressed loops are indicated by a partial oval ending in a cross around a hole, and the proposed ablation line is indicated by a dotted line between two holes.

FIG. 3 illustrates the simulation of induced reentry around the RPV with a suppressed loop around the MV.

FIG. 4 illustrates the simulation of FIG. 3, whereby an ablation between the RPV and LPV has taken place. The reentry loop around the RPV terminates, but the suppressed loop around the MV takes over and evolves into a full reentry loop.

FIG. 5 illustrates the simulation FIG. 4, but with the addition of an ablation between the RPV and MV. The loop around the MV is now ablated and has no path to complete its trajectory, and there are no other (suppressed) loops present. As a result of this, the tachycardia terminates.

FIG. 6 illustrates the arrhythmia in clinical case NC3, as illustrated in the example section.

FIG. 7 illustrates the arrhythmia in clinical case OC29, as illustrated in the example section.

FIG. 8 illustrates all mathematical possibilities for a topological sphere with three holes.

FIG. 9 also illustrates all mathematical possibilities for a topological sphere with three holes, taking into account equivalency.

FIG. 10 illustrates all mathematical possibilities for a topological sphere with four holes, taking into account equivalency.

FIG. 11 illustrates a suppressed loop of around 60%, as identified in an embodiment according to the present invention.

FIG. 12 illustrates how an atrium with non-conducting tissue, e.g., a scar at the anterior wall, may be topologically classified as a sphere with 4 holes.

FIG. 13 illustrates examples of a full reentry loop (FIG. 13A), a suppressed loop (FIG. 13B), and forks (FIG. 13C and FIG. 13D).

FIG. 14 illustrates patterns of a wave travelling around a hole.

FIG. 15(a) illustrates mathematical combinations for 3 hole cases of true and suppressed loops with at least one true loop. The index theorem excludes many possibilities, as indicated by a cross instead of a check mark. FIG. 15(b) illustrates clinical counterparts of the allowed cases of FIG. 15(a) when taking into account all permutations over different anatomical holes. FIG. 15(c) illustrates valid options for 4 hole cases with 2 or 4 loops. Rotations around 2 holes simultaneously have been excluded.

FIG. 16 illustrates the assignment of indexes for the 2-hole, 3-hole and 4-hole cases, as exemplified in Example 7.

### DETAILED DESCRIPTION OF THE INVENTION

As used below in this text, the singular forms “a”, “an”, “the” include both the singular and the plural, unless the context clearly indicates otherwise. The terms “comprise”, “comprises” as used below are synonymous with “including”, “include” or “contain”, “contains” and are inclusive or open and do not exclude additional unmentioned parts, elements, or method steps. Where this description refers to a product or process which “comprises” specific features, parts, or steps, this refers to the possibility that other features, parts or steps may also be present, but may also refer to embodiments which only contain the listed features, parts, or steps.

The terms first, second, and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein. The terms top, under, and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

The enumeration of numeric values by means of ranges of figures comprises all values and fractions in these ranges, as well as the cited end points. The term “approximately” as used when referring to a measurable value, such as a parameter, an amount, a time period, and the like, is intended to include variations of +/−10% or less, preferably +/−5% or less, more preferably +/−1% or less, and still more preferably +/−0.1% or less, of and from the specified value, in so far as the variations apply to the invention disclosed herein. It should be understood that the value to which the term “approximately” refers per se has also been disclosed. Percentages as used herein may also be noted as dimensionless fractions or vice versa. A value of 50% may for example also be written as 0.5 or ½.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous. Reference throughout this specification to “one embodiment”, “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention, and form different embodiments, as would be understood by those in the art. For example, in the appended claims, any of the claimed embodiments can be used in any combination. In the description provided herein, numerous specific details are set forth. However, it is understood that embodiments of the invention may be practiced without these specific details. In other instances, well-known methods, structures, and techniques have not been shown in detail in order not to obscure an understanding of this description.

Throughout this disclosure, various publications, patents, and published patent specifications may be referenced by an identifying citation. All documents cited in the present specification are hereby incorporated by reference in their entirety. In particular, the teachings or sections of such documents herein specifically referred to are incorporated by reference. All references cited in this description are hereby deemed to be incorporated in their entirety by way of reference.

Unless defined otherwise, all terms disclosed in the invention, including technical and scientific terms, have the meaning which a person skilled in the art usually gives them. For further guidance, definitions are included to further explain terms which are used in the description of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

As used herein, the terms “AT” or “atrial tachycardia” refer to a type of regular heart rhythm problem (arrhythmia) originating from the upper chambers (atria) of the heart, rather than from the sinoatrial node, the normal origin of the heart's electrical activity. The underlying mechanism can be either the rapid and regular discharge of an abnormal focus; or the presence of a reentry movement around an obstacle in the atria like a valve, the veins, or some scar tissue (anatomical reentry). As used herein, the terms “VT” or “ventricular tachycardia” refer to a fast but regular heart rate arising from the lower chambers of the heart. Similarly, a regular focal source or stable anatomical reentry are the source of a VT.

As used herein, the term “LA” refers to the “left atrium” while the term “RA” refers to the “right atrium”. There are two atria in the human heart—the left atrium receives blood from the pulmonary circulation, and the right atrium receives blood from the vena cavae of the systemic circulation. During the cardiac cycle, the atria receive blood while relaxed in diastole, then contract in systole to move blood to the ventricles. Topology is the mathematical study of the properties that are preserved through deformation, twisting, and stretching of objects.

As such, the left atrium (LA) may be viewed as a sphere with 3 holes, namely the left pulmonary vein (LPV), the right pulmonary vein (RPV), and the mitral valve (MV). For the right atrium, the openings are the tricuspid valve (TV), the inferior vena cava (IVC) and the superior vena cava (SVC). Depending on the patient, the LPV can be viewed as a single obstacle (hole), but sometimes there is conduction between left superior pulmonary vein and the left inferior pulmonary vein. In these cases, one can view this as two different holes. Similar reasoning for the RPV Besides these natural obstacles, it is possible that the patient has additional non-conductive tissue in the form of scar tissue, which creates additional obstacles in the atria.

As used herein, the term “EP” refers to an electrophysiologist, also known as a cardiac electrophysiologist or cardiac EP. An EP is a cardiologist who focuses on testing for and treating problems involving arrhythmia. As used herein, the terms “ablation” or “cardiac ablation” refer to the use of heat or cold energy to create tiny scars in the heart to block irregular electrical signals and restore a typical heartbeat. The procedure is used to correct heart rhythm problems (arrhythmias).

As used herein, the terms “LAT” or “local activation time” refer to the time the cells in close vicinity of the electrode are activated. As used herein, the term “cycle length” refers to the time it takes for a regular arrhythmia to find itself in the same position.

As used herein, the term “reentry loop” refers to a fully closed circuit of an electrical wave in the heart. A reentry loop, also referred to as a true reentry loop, may also be referred to as a dominant loop.

As used herein, the term “suppressed loop” refers to an almost but not fully closed reentry loop which can lead to a new slower arrhythmia when not ablated. Typically, a suppressed loop becomes a fully closed reentry loop after the original reentry loop has been ablated. A suppressed loop may also be referred to as a bystander loop.

As used herein, the terms “directed network” or directed graph” are used interchangeably and refer to a network or graph that is made up of a set of vertices connected by directed edges.

In a first aspect, the present invention relates to a computer-implemented method for identifying possible ablation lines. The method preferably comprises the steps of:
a) receiving spatiotemporal electrophysiological data of a subject's heart;
b) converting the spatiotemporal electrophysiological data into converted data; preferably wherein the converted data comprises a directed graph;
c) identifying one or more reentry loops from the converted data; preferably from the directed graph;
d) identifying one or more suppressed loops from the converted data; for example from the directed graph; and,
e) identifying a set of one or more possible ablation lines that terminate the one or more reentry loops and the one or more suppressed loops.

Steps a) to c) may be performed using the method as described in patent application WO 2019/105986 A1, hereby enclosed by reference in its entirety. Preferred embodiments of the steps and/or system features in WO 2019/105986 A1, are also preferred embodiments of the steps and/or system features in the present invention.

Steps a) and b) may be considered pre-processing steps. In some embodiments, the method comprises the steps of:
ab) receiving converted spatiotemporal electrophysiological data of a subject's heart; preferably wherein the converted data comprises a directed graph;
c) identifying one or more reentry loops from the converted data; preferably from the directed graph;
d) identifying one or more suppressed loops from the converted data; for example from the directed graph; and,
e) identifying a set of one or more possible ablation lines that terminate the one or more reentry loops and the one or more suppressed loops.

Preferably, steps c) and d) are performed simultaneously.

In a second aspect, the present invention relates to a system or device for identifying ablation lines. The system or device preferably comprises:
an input unit configured for receiving spatiotemporal electrophysiological data of a subject's heart;
a mapping unit and a directed graph generator configured for converting the spatiotemporal electrophysiological data in a directed graph; and,
a topological feature analyser for determining features in the directed graph;
   wherein the topological feature analyser is configured for identifying reentry loops as well as suppressed loops.

In some embodiments, the system comprises:
an input unit configured for receiving converted spatiotemporal electrophysiological data of a subject's heart; preferably wherein the converted data comprises a directed graph;
a topological feature analyser for determining features in the converted data; preferably in the directed graph;
   wherein the topological feature analyser is configured for identifying reentry loops as well as suppressed loops.

In some preferred embodiments, the system or device according to the second aspect of the invention, and (preferred) embodiments thereof, is configured to perform the computer-implemented method according to the first aspect of the invention, and (preferred) embodiments thereof.

A first step of the method preferably comprises receiving spatiotemporal electrophysiological data, preferably an excitation pattern of the electrical activity. Preferably the spatiotemporal electrophysiological data was gathered by a plurality of electrodes. The plurality of electrodes may correspond to a plurality of spatial locations in or on the heart. The method preferably comprises providing, for each of the plurality of spatial locations, a plurality of time values indicative of a time of occurrence of a predetermined feature of a plurality of electric potential waveforms at the spatial location. A time feature extractor may provide, for each of the plurality of spatial locations, a plurality of time values indicative of times of occurrence of a predetermined feature of a corresponding plurality of electric potential waveforms at that spatial location, based on said received spatiotemporal electrophysiological data.

The system or device preferably comprises an input unit for receiving spatiotemporal electrophysiological data, preferably an excitation pattern of the electrical activity, e.g., gathered by a plurality of electrodes, corresponding to a plurality of spatial locations in or on the heart. The system or device preferably comprises a time feature extractor for providing, for each of the plurality of spatial locations, a plurality of time values indicative of times of occurrence of a predetermined feature of a corresponding plurality of electric potential waveforms at spatial location, based on said received spatiotemporal electrophysiological data.

In a system or device in accordance with embodiments of the present invention, the input unit may be adapted for receiving the spatiotemporal electrophysiological data comprising, for each of the plurality of spatial locations, the at least one time value indicative of the time of occurrence of the predetermined feature of the at least one electric potential waveform, and the feature extractor may be adapted for providing the at least one value for each of the plurality of spatial locations from the received spatiotemporal electrophysiological data.

Each electric potential waveform may refer to the electrical activity associated with a single heart pulse. For example, the plurality of electric potential waveforms may be organized as an electric signal trace, e.g., a voltage as function of time, which comprises a sequence of separate pulses, e.g., referred to as the waveforms. The predetermined feature may be substantially the same feature that is detected in each of the waveforms and in each of the spatial locations. The predetermined feature may be an activation time, but embodiments of the present invention are not necessarily limited thereto, e.g., the feature may be a deactivation time or another waveform feature. The predetermined feature may be determined by a morphological classification representative of the waveform shape for each of the plurality of electric potential waveforms, e.g., in each location and for each heart pulse.

In some preferred embodiments, the spatiotemporal electrophysiological data received in step a) comprises spatial coordinates, and optionally local activation times of electrodes. In some preferred embodiments, the spatiotemporal electrophysiological data received by the input unit comprises at least the spatial coordinates, and optionally the local activation times of the electrodes. The system or device may comprise the plurality of electrodes.

In a subsequent phase, the spatiotemporal electrophysiological data, preferably the excitation pattern of the electrical activity, most preferably the spatial coordinates, and optionally the local activation times of the electrodes, are preferably converted in a directed graph.

Preferably this step is performed by directed-graph mapping. In some preferred embodiments, step b) comprises directed graph mapping or DGM. Directed-graph mapping or DGM can automatically analyse any type of arrhythmia to find reentry. It preferably comprises the steps of converting the excitation pattern of the electrical activity in a directed graph; and analysing this directed graph. DGM can be used for the analysis of clinical data, experimental data, and/or in-silico data. DGM is based on transforming a given dataset of an arrhythmia into a directed graph. It only requires a file including the spatial coordinates and the local activation times (LATs) of the electrodes. The local activation times can be extracted from the measured signals. There is no need for any regularity of the spatial locations of the dataset, making it independent of the measuring system. Therefore, it can take as input any extracted data from a computational dataset, experimental dataset like needle data, socket data, . . . , or from a clinical dataset, grid electrode data, basket catheter data, etc. The spatial locations of the electrodes will be the nodes of the network.

The step of converting the spatiotemporal electrophysiological data in a directed graph preferably comprises providing pairs of adjacent spatial locations of the plurality of spatial locations. The step of converting the spatiotemporal electrophysiological data in a directed graph may also comprise associating with each determined pair of adjacent spatial locations a distance between the pair of adjacent spatial locations. The step of converting the spatiotemporal electrophysiological data in a directed graph preferably comprises generating a directed graph comprising directed edges, each directed edge connecting a pair of the pairs of adjacent spatial locations in a sense of direction that is representative of a direction of propagation in time of at least one electric potential waveform of the plurality of electric potential waveforms between the pair of spatial locations. At least one time value of the plurality of time values for each spatial location of the pair may be taken into account to generate the directed graph, e.g., at least one time value for a first spatial location of the pair and at least one time of the other spatial location of the pair. Furthermore, a distance associated with the pair of spatial locations may also be taken into account to generate the directed graph.

In some embodiments, the local activation times (LATs) are not required. In some embodiments, the method and/or system merely requires the locations of the electrodes, and optionally a way to derivate the LAT. It has been found that the arrow in a directed graph may be derived without the need to determine the actual LAT. In some embodiments, the method comprises the direct step from two signals to a directed arrow by computing the correlation between the signals.

The system or device preferably comprises a mapping unit and/or preferably comprises a directed graph generator configured for converting the spatiotemporal electrophysiological data in a directed graph.

The system or device preferably comprises a mapping unit for providing pairs of adjacent spatial locations of the plurality of spatial locations. The mapping unit may also be adapted for associating a distance, between each pair of adjacent spatial locations, with each determined pair of adjacent spatial locations. In a system or device in accordance with embodiments of the present invention, the mapping unit may be adapted for storing the predetermined pairs of adjacent spatial locations and the distance between each pair of adjacent spatial locations in accordance with a predetermined spatial configuration of the plurality of electrodes.

The system or device preferably comprises a directed graph generator for generating a directed graph comprising directed edges, each directed edge connecting a pair of the pairs of adjacent spatial locations in a sense of direction that is representative of a direction of propagation in time of at least one electric potential waveform of the plurality of electric potential waveforms between the pair of spatial locations, in which at least one time value of the plurality of time values for each spatial location of that pair are taken into account, e.g., at least one time value of a first spatial location of said pair and at least one time value of a second spatial location, i.e. the other spatial location, of said pair. Furthermore, the directed graph generator may also take the distance associated with each pair of the pairs of adjacent spatial locations into account.

In some embodiments, the converted data, preferably a directed graph, is obtained through alternative means. As long as full (and optionally partial) cycles may be identified, true reentry loops (and optionally suppressed loops) may be identified. Links and/or cycles may be identified using other means as known by the skilled person in the art.

In FIG. 1, the preferred different sub-steps of the step of converting the spatiotemporal electrophysiological data in a directed graph according to some embodiments of the invention are shown. In some preferred embodiments, in a first sub-step, for each electrical sample, the neighbours are determined using the spatial coordinates of the data points, as illustrated in FIG. 1(A). A node may be considered a neighbour of another node if they are within a certain spherical distance. In some embodiments, the geometry of the atria/ventricles is taken into account when specifying neighbours. In some embodiments, the geometry of the atria/ventricles is not taken into account when specifying neighbours.

In a second sub-step, for a given time t, each node's first local activation time value larger than t may be selected. In case of a regular arrhythmia, it may be advantageous to add the cycle length to the given local activation time, as only one local activation time is given per node. Therefore, the correct cycle length is preferably provided. Then, for each node only a directed arrow is allowed to a neighbour if the spatial distance divided by the time difference in LATs is in between a certain allowed minimal and maximal conduction velocity, as illustrated in FIG. 1(B).

For standard parameter settings, the minimal conduction velocity may be set to 0.2 mm/ms and the maximal conduction velocity is set to 2.0 mm/ms. However for clinical cases, the software may choose its own minimum and maximal conduction velocity. For simulations, the user can adapt the conductions velocity bounds to accommodate the desired conduction in their situation. In this case, it may be preferred for the minimal conduction velocity to be within a range of 30% and 50% of the induced conduction of the simulation tissue and/or the maximal conduction velocity to be within a range of 150% to 200% of the induced conduction velocity. Repeating this process for all the nodes, this creates a single directed graph at a certain time t. In addition, a second network is created at a time t+δt, whereby δt is a parameter of the program, also referred to as the jump. This jump is preferably set to 40 ms in standard settings.

This second network is preferably merged with the first network as follows. If an additional arrow is created which has the same originating local activation time, it is added to the first network. This results in a full directed graph of excitation, as illustrated in FIG. 1(C). Notice that this step allows to create closed cycles in the network. As an intermediate step, the properties of this directed graph may be used to create more uniformly distributed nodes in case of regular datasets, as illustrated in FIG. 1(D). If the sub-step of FIG. 1(D) takes place, the sub-steps in FIG. 1(A)-(C) may be repeated.

The method preferably comprises the step of determining cycles in the converted data, preferably in the directed graph, and preferably comprises the step of outputting detected points and/or detected regions in or on the heart as representative of the cycles. According to the present invention, these cycles comprise both reentry loops as well as suppressed loops. The method optionally may also comprise determining graph sources and/or graph sinks in the directed graph and outputting detected points and/or detected regions in or on the heart as representative of the graph sources and/or graph sinks.

The system or device preferably comprises a topological feature analyser for determining features in the converted data, preferably in the directed graph. In some embodiments, the topological feature analyser is configured for identifying reentry loops as well as suppressed loops.

Optionally, the topological feature analyser may also be adapted for determining graph sources and/or graph sinks in the directed graph. In some embodiments, the system or device comprises an output for outputting detected points and/or detected regions in or on the heart as representative of the cycles detected by the topological feature analyser. In a system or device in accordance with embodiments of the present invention, the topological feature analyser may be adapted for determining the cycles in the converted data, preferably in the directed graph using a breadth-first search algorithm.

In the example of FIG. 1, once the final network is created, DGM searches for directed cycles. In the default visualization mode, a band of loops is created for clear visualization of the re-entrant pattern responsible for maintaining the arrhythmia, as illustrated in FIG. 1(F). DGM also computes a wavefront representing the excitation pattern, as illustrated in FIG. 1(E).

In complex cases, it remains challenging to find the correct mechanism of the AT. These are usually cases with additional scar tissue, slow conducting tissue and lines of block formed by previous ablations. Often patients who suffered previously from atrial fibrillation (AF) also have a complex anatomy in terms of scar tissue and ablation lines. Moreover, it is possible that multiple reentry loops are present simultaneously. In that case, two reentry loops can have the exact same cycle length, herein referred to as a “true double loop”. However, usually, one reentry loop has a shorter reentry cycle length and is thus faster than the other loop. In this case, the faster reentry may be called the “dominant loop” or “(true) reentry loop”, and the slower reentry cycle may be called the “bystander loop” or “suppressed loop”.

Entrainment mapping was introduced to find the dominant loop in case of doubt, as it is believed that the dominant loop should be the main ablation target. To find the dominant loop, the post-pacing interval (PPI) should not exceed the tachycardia cycle length (TCL) for more than 30 ms in the dominant circuit. It should be noted that entrainment mapping can be tedious as it can stop the AT, and is therefore not a standard technique used in the cath lab.

In the present invention, the method identifies reentry loops as well as suppressed loops. Preferably, the true reentry loops are related to a hole in the topology of the heart. In what is described herein, focus may be on the left atrium. However, it is understood that the present steps apply to the right atrium as well. One may also consider the right atrium as a sphere with holes. This can also be applied to the left ventricle and the right ventricle. Starting from a dataset containing points measured in the left atrium (x, y, z, and LAT) a network may be created in which reentry loops and suppressed loops are identified.

When 2 loops are found simultaneously, often one is dominant, while the other is a bystander. The difference between the two may be defined as follows: in case the bystander loop is ablated, the cycle length of the atrial tachycardia will not change. In case the dominant loop is ablated, on the other hand, the bystander loop will take over with an increased cycle length. Therefore, only ablating one loop, but not the other, does not solve the atrial tachycardia. In some cases, both reentry loops can be equally dominant. However, for the present invention, one does not necessarily need to make a distinction between dominant and bystander as both loops are preferably ablated anyway. Generally, the dominant loop will herein be referred to as a (true) reentry loop, while the bystander loop will be referred to as a suppressed loop. The true reentry loop is defined as a loop that is identifiable as a full cycle in DGM. Generally, this corresponds to having a green PPI-TCL when entrainment mapping is performed. In general, the EP will also agree with the true cycles found by DGM. However, to remove any ambiguity in the interpretation of colour maps, it is preferred to use DGM to annotate the true reentry loops.

A suppressed loop may be defined as a loop that does not make a full rotation, but that takes over the AT if the true loops are ablated. A suppressed loop is preferably defined as a loop that is identifiable as a partial (incomplete) cycle in DGM.

The method preferably comprises the step of identifying reentry loops in the converted data, preferably the directed graph, and preferably comprises the step of outputting detected points and/or detected regions in or on the heart as representative of the reentry loops. To uncover the reentry loops in the arrhythmia, one can search for closed cycles in the directed graph, for example with a Breadth First Search algorithm. Preferably, only the loops which have the best variance are identified. In some embodiments, the reentry loops which have the best variance are identified using the following algorithm:
ordering the values of the local activation times for each consecutive node of the cycle on the Y-axis of a graph:
drawing a straight line between the first and last node;
calculating the variance as the sum of the quadratic errors between the line and the actual local activation times; and,
visualising the loops with the lowest variance.

In case multiple cycles are detected, one may calculate the geometrical mean of the cycle. If the geometrical means of 2 cycles differ more than the value of the parameter core separator, these re-entries may be visualized separately. To enhance the intuitive visualization and corresponding interpretation, a loop-band feature may be added, which emphasises and colours the reentry loops, as illustrated in FIG. 1(F).

The method preferably comprises the step of identifying suppressed loops in the converted data, preferably in the directed graph, and preferably comprises the step of outputting detected points and/or detected regions in or on the heart as representative of the suppressed loops.

A suppressed loop may be defined as a loop that can be induced or as a loop that can take over the arrhythmia if the “conventional” reentry loops are ablated, while the suppressed loop is not ablated. Therefore, an optimal ablation strategy consists of cutting all loops: all reentry loops and all suppressed loops. By ablating for example a common region (usually called the isthmus) of a “conventional” reentry and suppressed loop, the physician terminates the current tachycardia and prevents the second tachycardia from taking over all in one single procedure, removing the need of a second procedure.

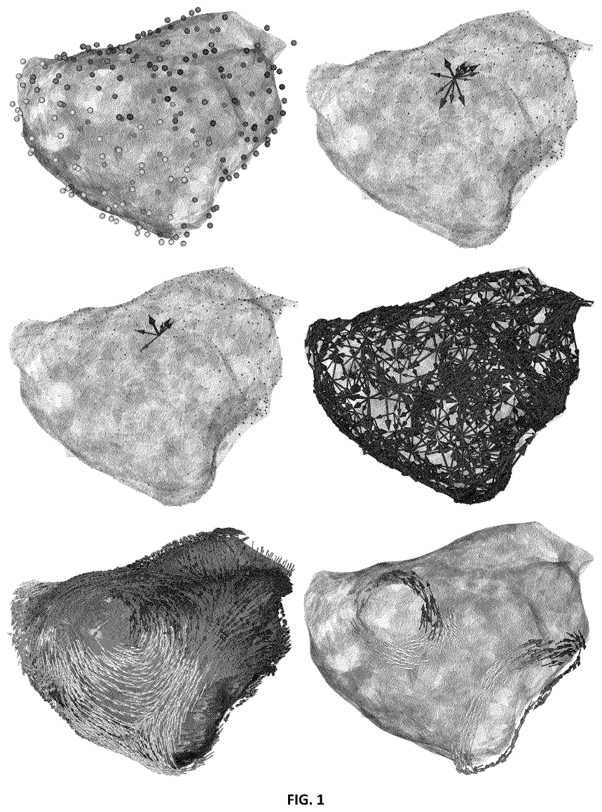

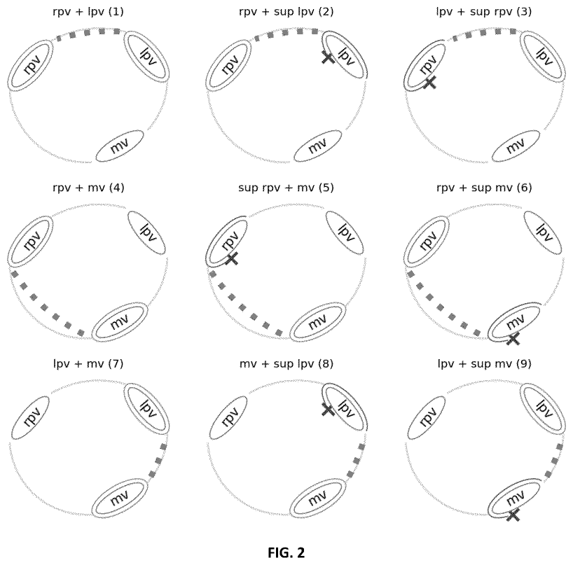

A suppressed loop stops continuing when its wavefront collides with another wavefront before its cycle can be completed. This other wavefront originates from the “conventional” reentry loop that is present in the system. When ablating the “conventional” reentry loop (full ovals in FIG. 2), the physician eliminates the wave that collides with the suppressed loop. The suppressed loop (partial ovals with a cross in FIG. 2) will then be able to continue its course and complete the cycle. This situation then result in a new atrial tachycardia and the physician will have to spend additional time on the same patient.

In some embodiments, the suppressed loops are identified purely visually, for example by visually interpreting the wavefront that is being output by a directed graph, for example by DGM. In some embodiments, the suppressed loops are identified automatically. This allows for the detection of the suppressed loops to occur more objectively as to not indulge biases. In some preferred embodiments, step d) comprises visual identification of the suppressed loops by a user. In some preferred embodiments, step d) comprises automatic identification of the suppressed loops by the computer. In some embodiments, the automatic identification of suppressed loops is based on the directed graph. In some embodiments, the automatic identification of suppressed loops is not based on a directed graph.

In some preferred embodiments, in step d) a suppressed loop is defined as a partially complete loop with a threshold of completion, whereby the threshold is for example at least 50%, for example at least 60%, for example at least 65%, for example at least 70%, for example at least 75%.

An algorithm to obtain a suppressed loop (and to define the threshold of completion) is provided in Example 4. Preferably, the suppressed loop(s) are identified from spatial and temporal data, preferably converted into a mesh, optionally via triangulation. Triangulation may only be required if the nodes on the edges of the holes are not yet obtainable from the metadata. For some mapping systems and tools, this information is already available in the standard fields of the data. In some embodiments, the suppressed loop(s) are identified by detecting edges of holes, such as anatomical holes and/or non-conductive tissue. In some embodiments, the suppressed loop(s) are identified by analysing LATs around one or more holes, preferably by adding a direction of wave propagation. In some embodiments, the suppressed loop(s) are identified by determining how far a wave circumvents a hole in both directions.

In some preferred embodiments, the angle of the greatest distance may be calculated to determine how far the loop is completed. If this angle is above a certain threshold (e.g. over 60% or 213 degrees), the loop may be defined as a suppressed loop. Such a loop will typically take over when only the true reentry loops are ablated. Using the same algorithm, if the angle amounts 360° or 100%, the loop may be defined as a true reentry loop.

In some preferred embodiments, step e) comprises classifying the identified loops prior to identification of the ablation lines. The possible ablation lines are preferably a result of the classification. This way, a unique classification may be obtained based on the anatomy of the patient for each AT. This classification may give rise to a unique ablation strategy which in theory can be applied to any given regular AT.

The classification may be obtained by extending an existing software package, such as Directed-Graph Mapping (DGM), which can automatically analyse any type of arrhythmia to find reentry or focal sources; wherein the software is extended so it can find not only the reentry loops, but also the suppressed loops. Preferably, the topology of the atria is used to create a unique classification for AT.

In general, topology is concerned with the properties of a geometric object that are preserved under continuous deformations, such as stretching, twisting, crumpling, and bending. However, one cannot close or open holes, tear, or glue to the object.

When looking at the left atrium, with no scar-tissue present and taking the RPV as a single vein as well as the LPV, one may consider the left atrium as a sphere with three holes: LPV, RPV and MV. In FIG. 2, all cases with two loops (reentry and suppressed) present around two of the given holes with at least 1 reentry loop will be discussed. Moreover, we can continuously deform the atrium into a sphere with 3 holes, without changing the topology.

Scar tissue or non-conductive tissue can add holes to the sphere, while ablation lines (or also called lines of block) connect two holes and therefore reduce the two connected holes to a single hole.

To describe anatomical reentry, the anatomy of the left and the right atrium presents 3 natural openings or holes. For the left atrium, the three openings are the mitral valve (MV), the left pulmonary veins (LPV) and the right pulmonary veins (RPV). For the right atrium, the openings are the tricuspid valve (TV), the inferior vena cava (IVC) and the superior vena cava (SVC). Besides these natural obstacles, it is possible that the patient has additional non-conductive tissue in the form of scar tissue, which creates additional obstacles in the atria. Therefore, anatomical reentry may be divided in two types: (1) one can have macro-reentry around one of the 6 anatomical larger obstacles as described above namely the MV, LPV, RPV, TV, IVC, SVP, and (2) reentry can occur around scar tissue. When the scar tissue is small, this is often called micro-reentry.

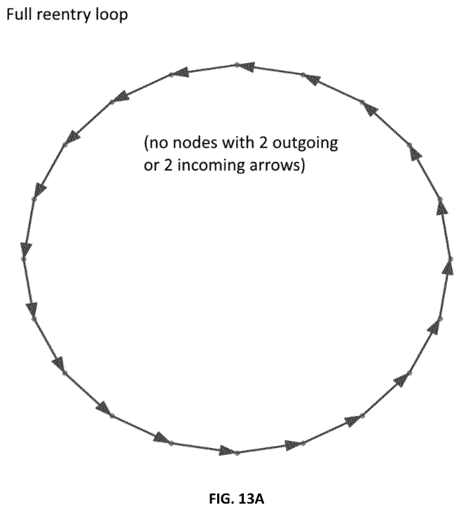

To uniquely classify the AT, it is preferred to identify the topology, i.e., to identify the number of holes for the specific anatomy of the subject. For example, a subject with scar tissue at the anterior wall of the left atrium, and further no ablation lines, would have 4 holes, as illustrated in FIG. 12. For each hole, there are now three possibilities. Around each hole, there can be a true reentry loop, there can be a suppressed loop, or no reentry loop. From the moment there are 4 or more holes present, we can repeat this process by taking together two holes. So around two holes, one can also have a true reentry loop, a suppressed loop, or no reentry loop. In the case of three holes, having a reentry around two holes, is equivalent to having a reentry around the remaining hole.

FIG. 9 illustrates the possibilities arising in the case of three holes. First, a single true reentry loop may be rotating around any of the three holes. Second, two true reentry loops, also called a double loop reentry, may rotate around any 2 chosen holes. Third, a true reentry loop may occur in combination with a suppressed loop. There are more linear combinations possible, but these are clinically impossible or not relevant. FIG. 9 only illustrates the 3 clinically relevant combinations out of 10 possibilities.

This way, the classification of a regular AT may be generalised. After determining the number of anatomical obstacles or holes in the left or right atrium, the true reentry loop(s) and the suppressed loop(s) need to be identified. In general, each loop can rotate around any number of holes simultaneously, taking into account that this is similar to a rotation (in the opposite direction) around the remaining obstacles.

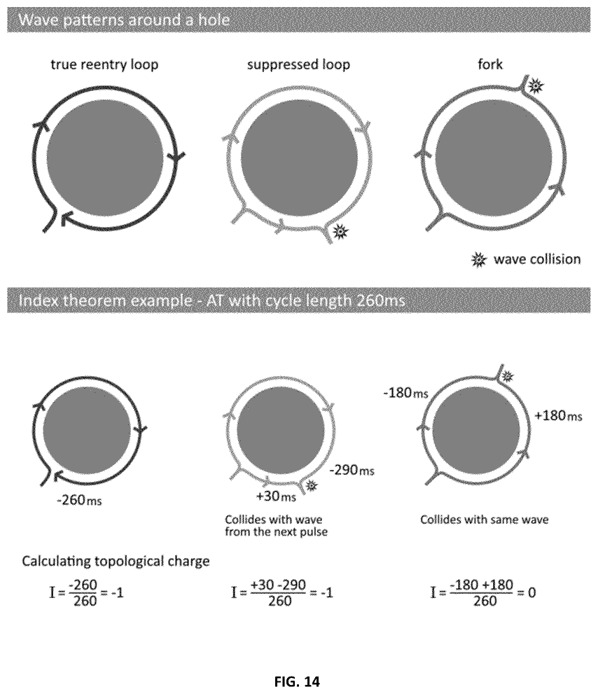

In some embodiments, the method comprises the step of using the index theorem to identify loops. The index theorem states that the sum of topological charges of all loops on a closed surface (like a sphere) with a finite number of holes should be zero. This implies that for every clockwise loop, a counterclockwise loop needs to exist. In other words, loops will always come in pairs of two. In some embodiments, the method comprises the step of using the index theorem to discount mathematically impossible and/or unlikely loops. In some embodiments, the method comprises the step of using the index theorem to find accompanying clockwise/counterclockwise loops. Using the index theorem has the advantage that loops can be identified more quickly and more efficiently. The index theorem has further the advantage that it provides a very general way to indicate a loop. The loop can be true or suppressed. While the index theorem as such may not make a distinction between a true and a suppressed loop, as they both will have an index of +1 or −1, in FIG. 14 it is demonstrated that, for a true loop, the LATs around a hole will increase and taking the sum of the differences between subsequent points will give you the entire cycle length. For a suppressed loop, differences of LATs will be larger than the cycle length in one direction (e.g. 30 ms), to cancel that additional part in the other direction (also 30 ms).

In some embodiments, the method comprises the step of identifying the topological charge/of a loop.

$$\sum I = 0$$

The topological charge/of a loop may be defined by how far the rotation goes around the hole, with −1 or −2π being a full clockwise rotation, depending on normalization; and with +1 or +2π being a full counterclockwise rotation. This is similar to full rotations of −2 or +2π as the basis of the well-known phase mapping. Applying this to atrial tachycardia, the topological charge of a loop may be obtained by determining how far the wave travels around the hole in both directions, using negative numbers for clockwise and positive number for counterclockwise rotation and taking the sum of both numbers. After normalization using the cycle length, the resulting charge will be −1 or +1 in case of a full rotation and 0 otherwise. The advantage hereof is that a hole connected to a counterclockwise loop (topological charge +1), should always be connected to a hole connected to a counterclockwise loop (topological charge −1), which allows for more efficient identification of possible loops.

In FIG. 14 it is illustrated that this holds true for all possible patterns, with a true or suppressed loop adding a charge of −1 or +1, while a fork results in a charge of 0. The top panel in FIG. 14 illustrates patterns of a wave travelling around a hole. The bottom panel in FIG. 14 illustrates application of the Index Theorem for an AT with a cycle length of 260 ms. It some preferred embodiments, it is calculated how far the wave travels in both directions around the hole. Preferably, the result is subsequently normalised using the cycle length. In this example, clockwise rotation is negative while counterclockwise rotations is positive. Note that, in an idealised world with an infinite amount of accurate measurements, the resulting charge will be +1, −1 or 0. In the present invention, with typically only a discrete amount of measurements to describe a continuous wave, the resulting charge might vary slightly.

Taking any random number of points around the hole will provide an approximation of the topological charge. The more points, the more accurate the topological charge will be. Preferably, at least 8 points are used, preferably at least 10 points, preferably at least 12 points, preferably at least 15 points., we took between 15-100 points, it remains to be seen how many points we will need in clinical data for a good prediction of the topological charge. Nevertheless, it has been found by the inventors, that even with as little as 8 points around a hole, the distinction between a charge of 0 or +/−1 becomes very clear.

In some embodiments, a specific strategy is applied within the application of the index theorem, preferably for the 2-hole, 3-hole and/or 4-hole cases; whereby an index is assigned to each individual hole; preferably as exemplified above. The chosen index is preferably indicative of whether around that hole any true or suppressed loops are present or not. For example, each individual hole may be assigned an index of +1, −1 or 0; whereby if the index of the hole is +1 or −1, this means that around that hole, there is either a true or a suppressed loop.

For the 2-hole and/or 4-hole cases, it may also be possible to have rotation around more than one hole, for example around 2 holes. Therefore, in some embodiments, the combination of holes is also assigned an index. For example, the index of the 2 holes taken together can thus also be +1 or −1. Assuming it is +1, one of the holes will also have an index of +1, while the other hole will have an index of 0. This means that it will always be possible to assign indexes to the holes themselves. Preferably, the index is computed. Mathematically, the index of the outer loop should be equal to the sum of the indexes of the loops inside that outer loop. In addition, the total sum of the indexes around all the holes in the atrium of interest will then be zero. More generally, situations with more holes, such as 5 or 6 holes, might also have rotations around 3 holes or more. In such cases, the outer loop should again have the same index as the sum of the inner loops.

The present invention also relates to the construction of a database of a full mathematical classification for a sphere with three, four or more holes, for example representing the left atrium. Based on a full mathematical classification for a sphere with three holes, FIG. 2 may be extended as follows. Taking all possible combinations of reentry loops with suppressed loops, a database of 10 possibilities has been identified, whereby permutations are allowed over the different holes. In some embodiments, the method comprises the step of identifying the optimal ablation strategy based on such a database.

An optimal ablation strategy consists of preferably cutting all loops: true reentry loops and suppressed reentry loops. This may be performed by connecting the corresponding holes. Holes may be connected directly, or via another hole. How the actual ablation is performed in surgery might depend on other factors such as which EP or hospital, certain ablation lines are easier to perform than others. In some preferred embodiments, step e) comprises the step of identifying an isthmus common to a reentry loop and a suppressed loop.

Once the true reentry loop(s) and the suppressed loop(s) have been identified, an optimal ablation strategy may be to connect the holes in such a way that all true and suppressed loops can no longer exist. Depending on the anatomy and ablation strategy there can be multiple possible strategies to do this. However, preferably as few holes as possible should be connected to limit the number of ablation lines.

In some embodiments, an optimal ablation strategy comprises the step of searching for a minimal total ablation length, which can be easily computed with DGM. In some preferred embodiments, step e) comprises the step of identifying an ablation strategy comprising a minimal total ablation length. In some embodiments, using Dijkstra's shortest path algorithm, one may find the shortest path between any one node from one loop to any one node of another loop. In case of more than two loops, this process may be repeated for each combination of two loops and the shortest resulting combination is returned. In some preferred embodiments, step e) comprises the step of identifying a minimal number of ablation lines that terminate all identified reentry loops and suppressed loops.

In some embodiments, based on the index theorem exemplified above, the optimal ablation strategy comprises connecting a +1 hole with a −1 hole. If there is more than one +1 hole (and thus also more than one −1 hole), there is no particular preference to which +1 hole is connected to which −1 hole from a mathematical standpoint; and other practical preferences from the electrophysiologist may become more decisive.

However, depending on the electrophysiologist, different approaches may be taken. Some electrophysiologists prefer to never ablate the line between the RVP and MV, while others do ablate this line. Therefore, some electrophysiologists may prefer to ablate a larger area than the optimum for minimal total ablation length, as certain lines are never performed. Such a preference imposes constraints of areas that are not to be ablated. In some preferred embodiments, step e) comprises the step of imposing constraints of areas that are not to be ablated. Most preferably, minimally, all reentry loops and suppressed reentry loops should be ablated.

In some preferred embodiments, the method comprises the step of consulting the optimal ablation strategy in a topological database. Preferably, the topological database proposes, for a certain topology, which ablations might be successful. In some embodiments, the topological database collects, for a certain topology, which previously performed ablations were successful and which ablations were unsuccessful. In such an embodiment, if two cases have the same topology, one could see which possible ablations were successful on these cases. Preferably, in the database, all loops and suppressed loops are cut.

In some embodiments, the method is a computer-implemented method. In some embodiments, one or more steps of the method are performed by a computer. In some embodiments, all steps of the method are performed by a computer.

The invention also relates to a system or device, such as a data processing apparatus, comprising means for carrying out one or more steps, for example all steps, of the method according to the first aspect of the invention, and (preferred) embodiments thereof. The system or device may comprise different hardware and/or software aspects to provide the functionalities as illustrated. For example, the system or device may comprise a computer, a computing system, or a processor, e.g., a general-purpose computing platform specifically programmed, e.g., by a suitable executable code, for implementing all or some elements described herein. In embodiments, the system or device may operate as a standalone device or may be connected, e.g., networked, to other machines in a networked deployment.

In a third aspect, the present invention relates to a computer program product directly loadable into the internal memory of a computer, or a computer program product stored on a computer readable medium, or a combination of such computer programs or computer program products, configured for performing a computer-implemented method according to the first aspect of the invention, and (preferred) embodiments thereof.

The invention also relates to a computer program product comprising instructions which, when the program is executed by a computer, cause the computer to carry out one or more steps, for example all steps, of the method according to the first aspect of the invention, and (preferred) embodiments thereof. The invention also relates to a computer-readable medium comprising instructions which, when executed by a computer, cause the computer to carry out one or more steps, for example all steps, of the method according to the first aspect of the invention, and (preferred) embodiments thereof. The invention also relates to a computer-generated image representative of reentry loops and suppressed loops identified by a method according to the first aspect, and (preferred) embodiments thereof.

The systems and methods as described herein may be used for any type of arrhythmia. In some preferred embodiments, the systems and methods are used for atrial tachycardia. In some preferred embodiments, the systems and methods are used for ventricular tachycardia, since it is equally possible to apply them to VT. For VT, the scar tissue may play the role of (additional) holes, since VT is usually maintained by re-entries around scar tissue. There are typically no anatomical obstacles.

In some preferred embodiments, the subject is a human subject.

In a fourth aspect, the present invention relates to use of the computer-implemented method according to the first aspect of the invention, and (preferred) embodiments thereof, or of the system or device according to the second aspect of the invention, and (preferred) embodiments thereof, for a subject suffering from atrial tachycardia, ventricular tachycardia, or atrial fibrillation; preferably atrial tachycardia or ventricular tachycardia; preferably atrial tachycardia.

In a fifth aspect, the present invention relates to computer readable storage medium comprising a topological database, the topological database preferably comprising ablation strategies corresponding to identified reentry loops and suppressed loops, suitable for use in the computer-implemented method according to the first aspect of the invention, and (preferred) embodiments thereof.

### EXAMPLES

The herein disclosed aspects and embodiments of the invention are further supported by the following non-limiting examples.

### Example 1

Four clinical cases may be associated to FIG. 2, illustrating that only ablating the true reentry loops, while not ablating the suppressed loops, leads to a slower atrial tachycardia, whereby the suppressed loop takes over. The 4 clinical cases consist of 2 cases of “Ipv+sup mv” and 2 cases of “rpv+sup mv” as presented in FIG. 2. The 4 clinical cases are therefore examples of the “3-hole cases”. In FIG. 2, the full cycles in greyscale denote a true reentry loop, the darker cycles marked with an X denote a suppressed loop, while the thick dotted lines denote the proposed ablation lines, cutting all true reentry and suppressed loops.

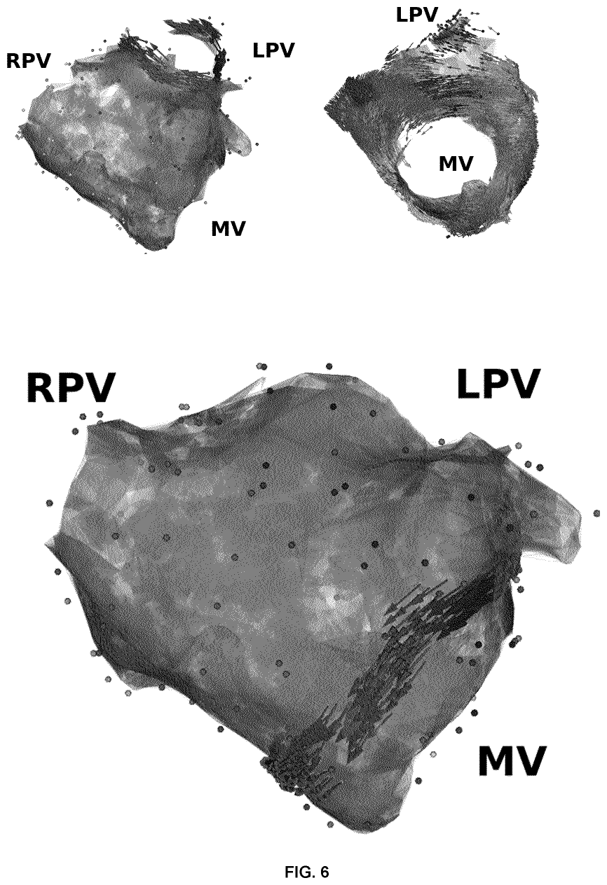

The first 2 cases (NC3 and NC4) conform to FIG. 2: Ipv+sup mv. In these 2 cases, DGM finds a true reentry loop around the LPV, and a suppressed loop around the MV. One of the cases (NC3) is shown in FIG. 6, while the other case is similar. On the top left subfigure, the loop around the LPV (before ablation) is shown. On the top right subfigure, the suppressed loop around the MV (before ablation) is shown. On the bottom subfigure, the loop around the MV (after ablation) is shown. This last figure confirms that the suppressed loop around the MV has taken over the arrhythmia.

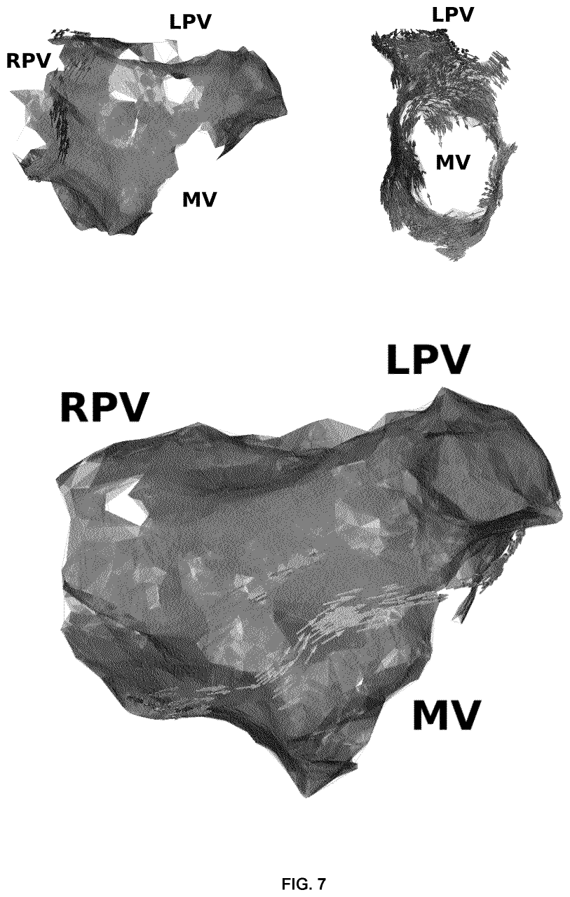

The 2 other clear clinical cases (OC29 and OC33) represent the class “rpv+sup mv” in FIG. 2. Here DGM found a true reentry circuit around the RPV and a suppressed loop around the MV. However, after ablating only the true loop around the RPV, the driving mechanism of the tachycardia changed to a loop around the MV. Case OC29 is shown in FIG. 7. On the top left subfigure, the loop around the RPV (before ablation) is shown. On the top right subfigure, the suppressed loop around the MV (before ablation) is shown. On the bottom subfigure, the loop around the MV (after ablation) is shown. This last figure confirms that the suppressed loop around the MV has taken over the arrhythmia.

### Example 2

In this simulated example, a model of a left atrium was used, based on data of real patients. This model of the left atrium was then split into parts to change the conduction velocity of certain regions. A monodomain Courtemanche cell model was used for simulating cell potentials. The Sodium conductivity was set to 3.9, the Calcium conductivity was set to 0.03095, and the Potassium rapid delayed rectifier conductivity was set to 0.2352. All other conductivity values were left at standard values. Other cardiac cell models with different parameters can be used as long as these parameters allow the development of re-entrant activity (formation of loops) in the anatomical model.

A stimulus was induced in the system (depending on the reentry circuit is aimed at) to jump start the electrical wave, and the simulation ran until equilibrium. Next, the simulation was paused, and an ablation line was induced, blocking conductivity through that region. These ablation lines were based upon the ones that electrophysiologists perform in the operation room. Finally, the simulation was resumed, and the evolution of the tachycardia was evaluated.

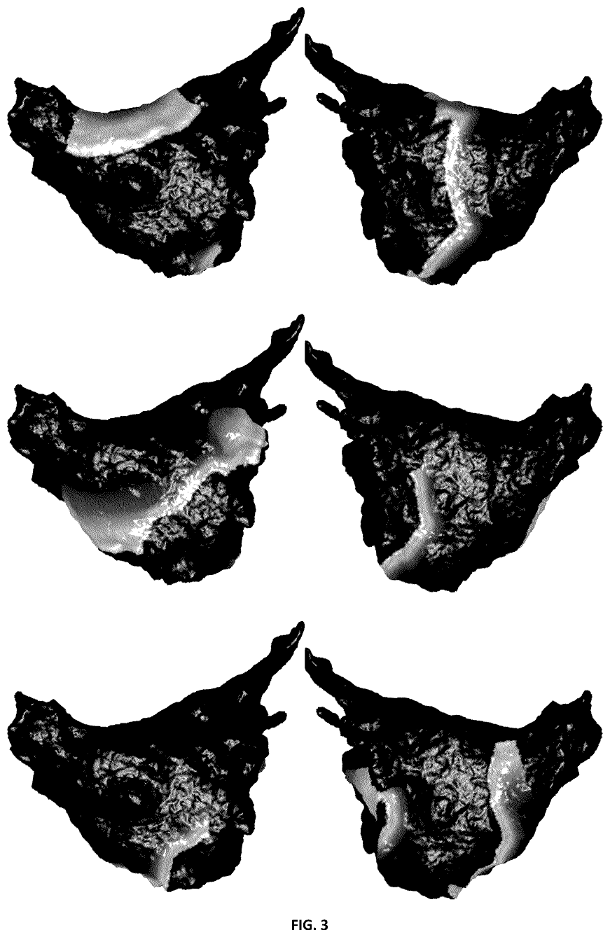

FIG. 3 illustrates the simulation of an atrial tachycardia with a reentry loop around the RPV (DGM clearly shows the reentry loop) and a suppressed loop around the MV. This atrial tachycardia has a cycle length of 215 ms.

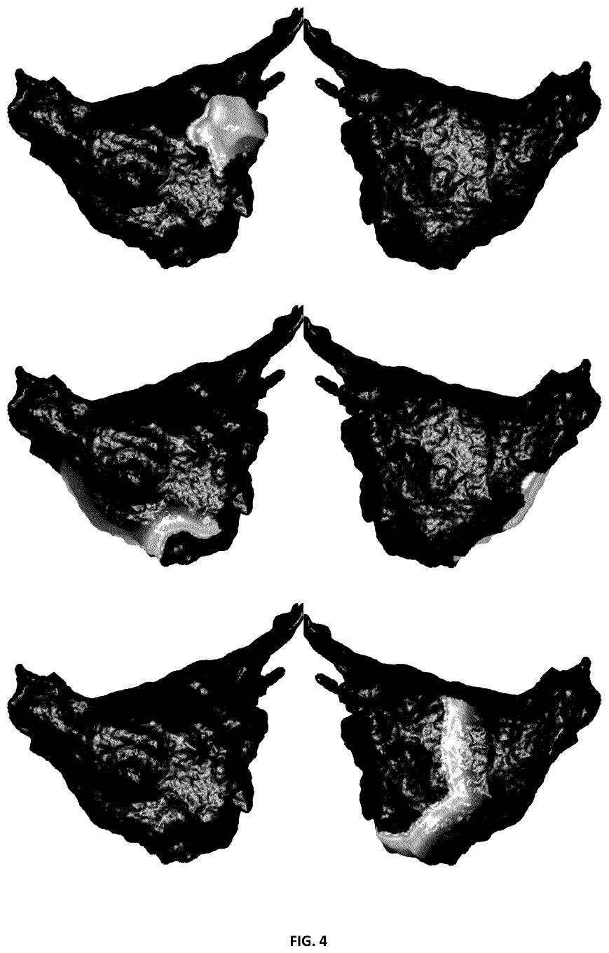

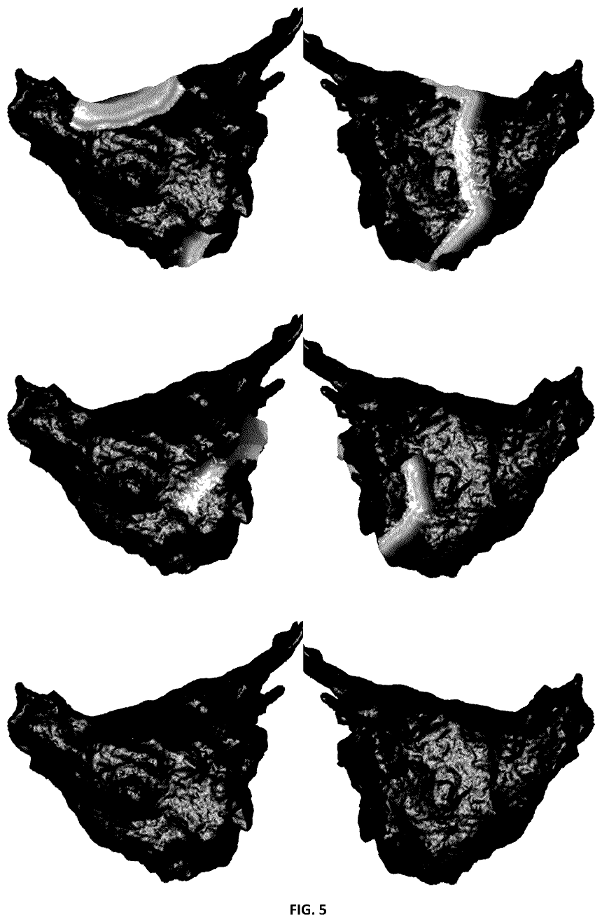

Upon only ablating the area between the RPV and the LPV, the reentry loop around the RV terminated and the suppressed loop evolved into a new reentry loop around the MV. Moreover, the cycle length increased with 30 ms to a total of 245 ms, as illustrated in FIG. 4. However, after ablating the anterior wall (the line between RPV and MV), the reentry loop around the RV terminated, but simultaneously the suppressed loop around the MV had no path to complete its trajectory. Using such an ablation instead, the atrial tachycardia stops, as illustrated in FIG. 5.

### Example 3

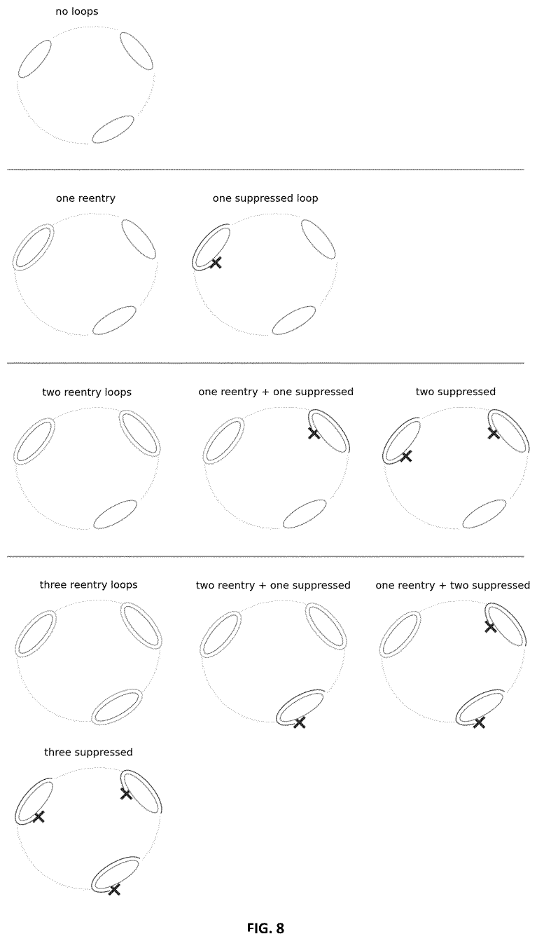

FIG. 8 illustrates all mathematical possibilities for a topological sphere with three holes.

FIG. 9 also illustrates all mathematical possibilities for a topological sphere with three holes, taking into account equivalency. Note that in the case of three holes, rotation around a single hole is equivalent with rotation around the 2 remaining holes, as illustrated at the top of FIG. 9. In this Figure, one can see the three possible combinations of true and suppressed loops in case of three holes in the chamber of interest. Only combinations that actually give rise to an arrhythmia (cases where at least one true loop is present) are illustrated in this Figure. A suppressed loop is shown in a dotted circle, while true reentry loops are shown in a full circle. The hypothesis of blocking the paths of all reentry circuits and all suppressed loops can be expanded upon to cases with more than 3 holes as well. Very often scar tissue is present which completely blocks wave propagation. Topologically, this creates an extra hole. This has an effect on the total number of topological possibilities. Even though the mathematical possibilities grow rapidly with each additional hole, the underlying idea of the invention remains. There are always only a limited number of possibilities for (combinations of) reentry loops and suppressed loops to occur. And thus only a limited number of possibilities to ablate each of them.

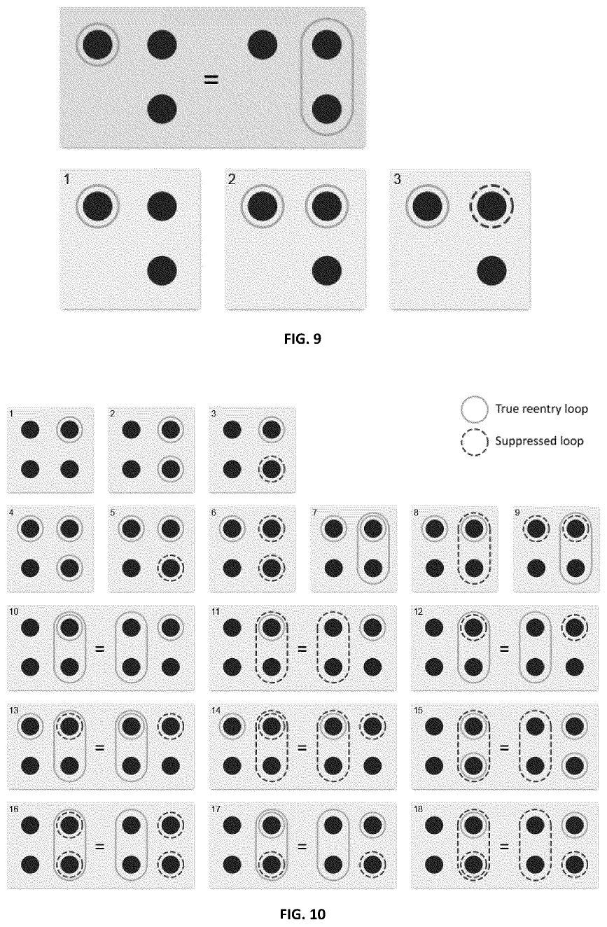

FIG. 10 illustrates all mathematical possibilities for a topological sphere with four holes, taking into account equivalency. Note that in the case of four holes, rotation around 2 holes is equivalent with rotation around the 2 other holes, similar to what is shown in FIG. 9. Other equivalencies are also illustrated in FIG. 10. In this Figure, we show all 17 possible combinations of true and suppressed loops in case of 4 holes in the chamber of interest. Only combinations that actually give rise to an arrhythmia (cases where at least one true loop is present) are illustrated in this Figure. A suppressed loop is shown in a dotted circle, while true reentry loops are shown in a full circle.

### Example 4

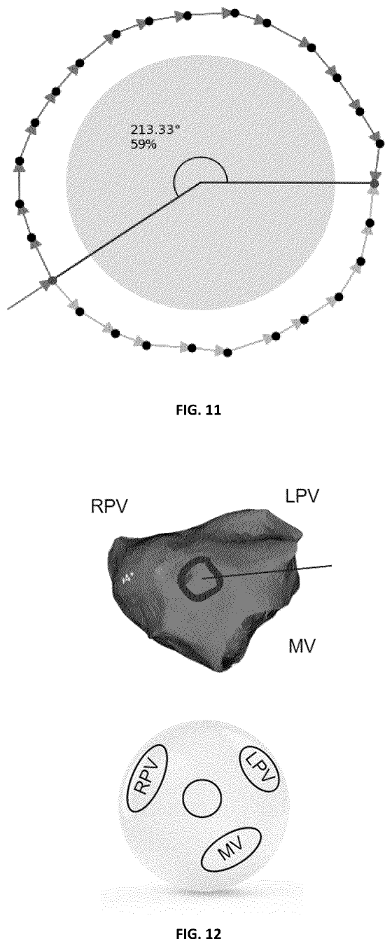

This example illustrates an exemplary algorithm to identify suppressed loops, though other algorithms may be used as well. FIG. 11 illustrates a suppressed loop of around 60%. Starting from the provided datapoints containing spatial and temporal data, one may proceed with the following steps to find the suppressed loop:
Starting from the provided datapoints, a mesh is constructed using Delaunay triangulation.
Using this triangulation, the edges of all holes are detected. Holes can be anatomical; as an example, for a healthy left atrium, the anatomical holes consist of the left pulmonary vein, right pulmonary vein, and mitral valve. In addition, non-conductive tissue may constitute additional topological holes in the substrate. To find the holes in the mesh, all triangles in the mesh may be considered. The triangles which have an edge that only belongs to a single triangle are at the edge of a hole. For each hole, collecting these edges will create a closed loop around that hole, as illustrated in FIG. 11.
For all nodes in the edges around a certain hole (thus the nodes on the border of that hole), the LAT is analysed. In case a node has no valid LAT yet, depending on the exact content of the datapoints, an LAT may be interpolated from nearby nodes.
Using the LAT on these nodes, an arrow may be drawn between every two consecutive nodes. The arrow is directed from the lowest LAT to the highest LAT, being the direction of the wave propagation.
Taking a node with two out-going arrows as starting point, it can be determined how far the wave circumvents the hole in both directions. This is indicated in FIG. 11.
The angle of the greatest distance may be calculated to determine how far the loop is completed. If this angle is above a certain threshold (e.g. over 60% or 213 degrees), the loop may be defined as a suppressed loop. Such a loop will typically take over when only the true reentry loops are ablated.

The triangulation steps above may only be required if the nodes on the edges of the holes are not yet obtainable from the metadata. For some mapping systems and tools, this information is already available in the standard fields of the data, and these steps may be skipped. If the information is not yet present in the metadata, it can be obtained by performing a triangulation.

While the above illustrated algorithm is particularly useful for identifying suppressed loops, it may also be used for identifying true reentry loops (for example as loops where the total angle is 360° or 100%, thereby indicating that all arrows circle around the hole in the same direction).

### Example 5

With regards to atrial fibrillation, some forms of AF may be considered irregular, complex AT. As described herein, true reentry loops and suppressed loops may also be identified for AF.

### Example 6

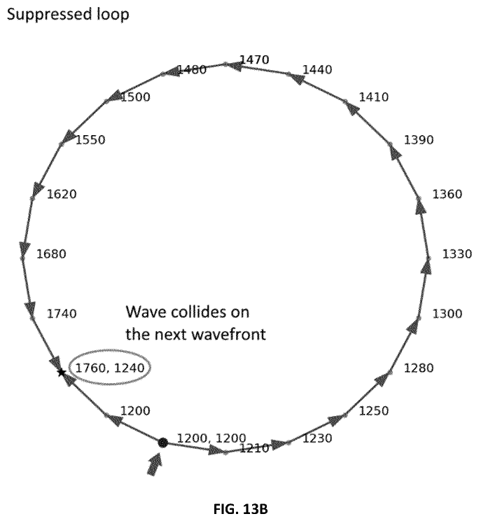

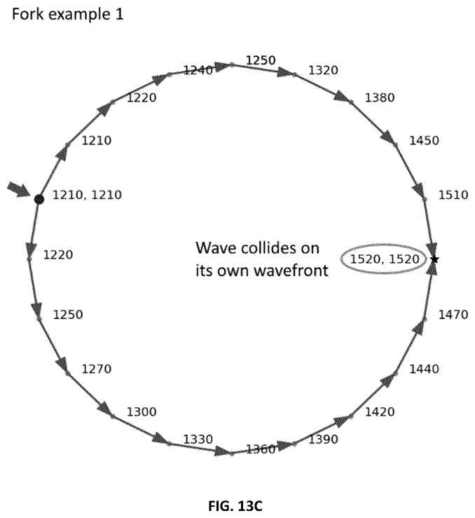

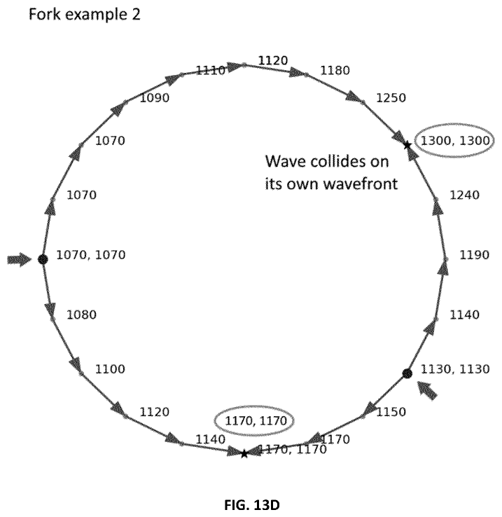

In some embodiments, forks may be identified. In FIG. 13, a further distinction may be made between suppressed loops and forks. A fork may occur when no reentry loops or suppressed loops are formed around holes, and may be defined as the wave simply splitting, thereby passing the hole on both sides. FIG. 13 illustrates examples of a full reentry loop (FIG. 13A), a suppressed loop (FIG. 13B), and 2 examples of forks (FIG. 13C and FIG. 13D).

In the case of a normal fork, the wave splits, goes around the hole and merges again with itself after passing the hole. It might be in more than two sections, but always colliding with the wavefront from the same pulse. This can be seen in Fork example 1 and Fork example 2 in FIG. 13C and FIG. 13D, as the LAT's of the wave coming from both sides are the same.

When we have a suppressed loop, as illustrated in FIG. 13B, on the other hand, the wave splits to go around the hole but collides with the wavefront of the next pulse. This is possible as the wave in the longer section has to travel further and/or pass through a region of slower conductivity. This can be seen in the suppressed loop in FIG. 13, as the LAT's of the wave coming from both sides differ in roughly one cycle length.

### Example 7

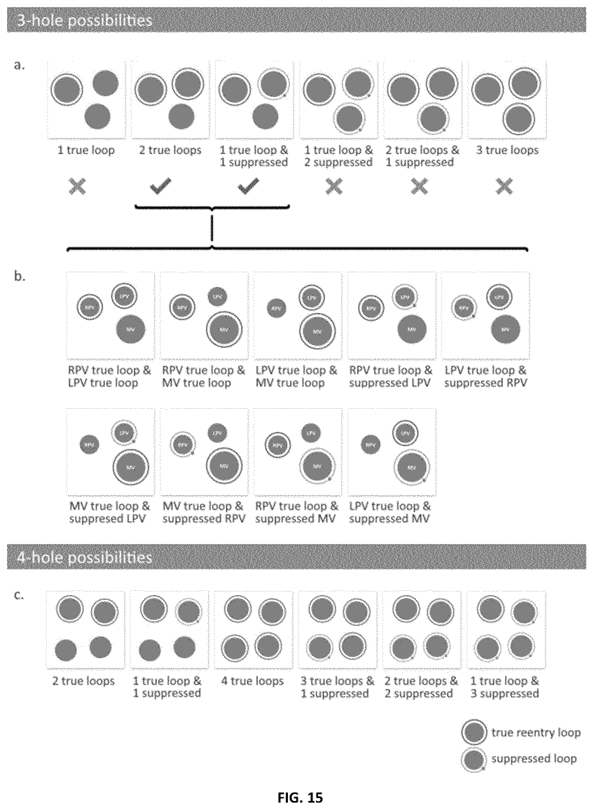

Various applications of the index theorem are exemplified in FIG. 15 and below:
2-hole cases. For example, a 2-hole case may occur in the LA, in the absence of scar, when an ablation line already connected 2 holes like a roof line connecting the LPV and RPV. Connecting 2 holes merges them into one hole. Having only 2 holes creates a special case, as having a loop around the first hole, assuming it is a clockwise rotation, means that there will be a counterclockwise loop around the second hole. Therefore, in this case, the 2 re-entries are in fact the same, as one can continuously deform the first loop into the second, therefore turning a clockwise rotation into a counterclockwise rotation as one moves over the sphere. Thus, 2-hole cases are the only cases where both loops are topologically equivalent and can be considered to be the same loop. Nevertheless, the index theorem is still satisfied.
3-hole cases. The next scenario considers an anatomy with 3 holes. For example, a 3-hole case may occur in the LA, in the absence of dense scar tissue, having the LPV, RPV, and MV as 3 holes. Around any of the holes, one can have a true reentry loop, a suppressed loop, or a fork, as disclosed herein. One may infer all possible ATs by combining true and suppressed loops. Having a clockwise reentry around 1 hole is topologically equivalent with a counterclockwise reentry around the 2 remaining holes, see FIG. 15, panel a. As the index theorem needs to be satisfied, a single true reentry loop cannot exist. Therefore, combining the different possibilities for 3 holes, one may obtain 6 possible combinations of true and suppressed loops where at least one reentry is present, see FIG. 15, panel b. Moreover, as indicated by the index theorem, a clockwise rotation should be accompanied by a counterclockwise rotation. By considering the LA with the RPV, LPV and MV as topological holes, one may interfere 9 clinically different AT, presented in FIG. 15. However, by also distinguishing clockwise and counterclockwise rotations, one may find 18 different possible ATs for the 3-hole cases. Note that the distinction between true and suppressed reentry loops may be considered a continuous spectrum, as the difference may be defined by any arbitrary threshold, preferably a threshold of about 30 ms. Preferably, for clinical purposes, both the true and the suppressed loops are ablated in order to stop the AT. Therefore, making a clear distinction between true and suppressed loops may clinically not be highly relevant.
4-hole cases. For example, a 4-hole cases may occur in the LA, when there is a patch of non-conductive tissue (scar), in addition to the 3 anatomical holes. In the FIG. 15, panel c, all plausible configurations are illustrated for re-entries which occur around the holes separately. There are also many possible configurations with rotations around 2 holes simultaneously. Note that although the number of possibilities rapidly increases with the number of holes present, the number of possible loop configurations remains finite for any number of holes. N-hole cases. In general, non-functional reentry can be divided in two types: (1) reentry around one of the 6 larger anatomical obstacles—the MV, LPV, RPV, TV, IVC, SVC, and (2) reentry around non-conductive scar tissue. Both types can be true or suppressed. To generalise even further, reentry may also occur around more than 1 hole if at least 4 holes are present.

The inventors have surprisingly identified an optimized strategy within the application of the index theorem, more specifically for the 2-hole, 3-hole and 4-hole cases. For the 2-hole, 3-hole and 4-hole cases, they have found that it is possible to assign an index to an individual hole. Each individual hole will have an index of +1, −1 or 0. If the index of the hole is +1 or −1, this means that around that hole, there is either a true or a suppressed loop.

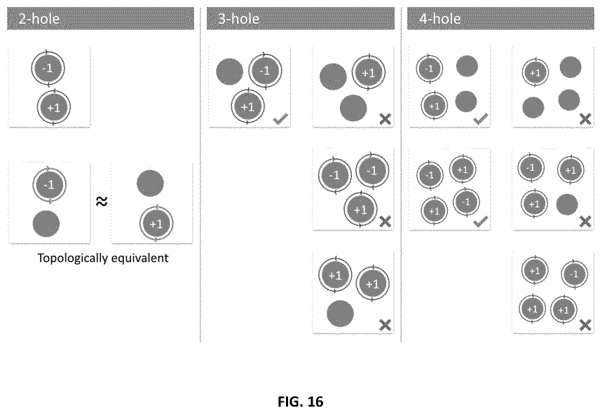

For the 2-hole and/or 4-hole cases, it is also possible to have rotation around more than one hole, for example around 2 holes. The index of the 2 holes taken together can thus also be +1 or −1. Assuming it is +1, one of the holes will also have an index of +1, while the other hole will have an index of 0. This means that it will always be possible to assign indexes to the holes themselves. The index of the outer loop will always be equal to the sum of the indexes of the holes inside that loop. In addition, the total sum of the indexes around all the holes in the atrium of interest will be zero. This is illustrated by the new topologies as identified in FIG. 16. More generally, situations with more holes, such as 5 or 6 holes, might also have rotations around 3 holes or more. In such cases, the outer loop should again have the same index as the sum of the inner loops.

With regard to the optimal ablation strategy, the inventors have found out that it consists of connecting a +1 hole with a −1 hole. As such, the inventors have found that only the holes themselves are important, not the loops around more than one hole. If there is more than one +1 hole (and thus also more than one −1 hole), there is no particular preference to which +1 hole is connected to which −1 hole.

1. A computer-implemented method for identifying possible ablation lines, the method comprising the steps of:
a) receiving spatiotemporal electrophysiological data of a subject's heart;
b) converting the spatiotemporal electrophysiological data into converted data;
c) identifying one or more reentry loops from the converted data;
d) identifying one or more suppressed loops from the converted data; and,
e) identifying a set of one or more possible ablation lines that terminate the one or more reentry loops and the one or more suppressed loops.

2. A computer-implemented method for identifying possible ablation lines, the method comprising the steps of:
ab) receiving converted spatiotemporal electrophysiological data of a subject's heart;
c) identifying one or more reentry loops from the converted data;
d) identifying one or more suppressed loops from the converted data; and,
e) identifying a set of one or more possible ablation lines that terminate the one or more reentry loops and the one or more suppressed loops.

3. The method according to claim 1, wherein the converted data comprises a directed graph, preferably obtained using directed graph mapping or DGM.

4. The method according to claim 1, wherein in step d) a suppressed loop is defined as a partially complete loop with a threshold of completion, whereby the threshold is preferably at least 50%.

5. The method according to claim 1, wherein step d) comprises visual identification of the suppressed loops by a user; and/or wherein step d) comprises automatic identification of the suppressed loops by a computer.

6. The method according to claim 1, wherein step e) comprises classifying the identified loops prior to identification of the ablation lines.

7. The method according to claim 1, wherein step e) comprises the step of identifying an isthmus common to a reentry loop and a suppressed loop.

8. The method according to claim 1, wherein step e) comprises the step of identifying an ablation strategy comprising a minimal total ablation length; and/or wherein step e) comprises the step of identifying a minimal number of ablation lines that terminate all identified reentry loops and suppressed loops; and/or wherein step e) comprises the step of imposing constraints of areas that are not to be ablated.

9. The method according to claim 1, comprising the step of consulting a proposed ablation strategy in a topological database.

10. A system or device for identifying ablation lines, the system or device comprising:
an input unit configured for receiving converted spatiotemporal electrophysiological data of a subject's heart;
a topological feature analyser for determining features in the converted data;
wherein the topological feature analyser is configured for identifying reentry loops as well as suppressed loops.

11. A system or device for identifying ablation lines, the system or device comprising:
an input unit configured for receiving spatiotemporal electrophysiological data of a subject's heart;
a mapping unit and a directed graph generator configured for converting the spatiotemporal electrophysiological data in a directed graph; and,
a topological feature analyser for determining features in the directed graph;
wherein the topological feature analyser is configured for identifying reentry loops as well as suppressed loops.

12. The system or device according to claim 10, configured to perform a computer-implemented method comprising the steps of:
receiving spatiotemporal electrophysiological data of a subject's heart;
converting the spatiotemporal electrophysiological data into converted data;
identifying one or more reentry loops from the converted data;
identifying one or more suppressed loops from the converted data; and,
identifying a set of one or more possible ablation lines that terminate the one or more reentry loops and the one or more suppressed loops.

13. A computer program, or a computer program product directly loadable into the internal memory of a computer, or a computer program product stored on a computer readable medium, or a computer program product comprising a non-transitory computer readable storage medium, or a combination of such computer programs or computer program products, configured for performing a computer-implemented method according to claim 1.

14. The computer-implemented method according to claim 1, wherein the subject is suffering from atrial tachycardia, ventricular tachycardia, or atrial fibrillation.

15. A computer readable storage medium comprising a topological database comprising ablation strategies corresponding to identified reentry loops and suppressed loops, suitable for use in the method according to claim 9.
